(19) United States
(12) Patent Application Publication
(10) Pub. No.: US 2023/0052662 A1
(43) Pub. Date: Feb. 16, 2023

(54) Antimicrobial Medical Devices and Methods of Forming Antimicrobial Medical Devices

(71) Applicant: LiViliti Health Products, Lake City, FL (US)

(72) Inventor: Brian Sharpe, Irmo, SC (US)

(21) Appl. No.: 17/879,895

(22) Filed: Aug. 3, 2022

Related U.S. Application Data
(60) Provisional application No. 63/236,103, filed on Aug. 23, 2021.
(60) Provisional application No. 63/229,707, filed on Aug. 5, 2021.

(51) Int. Cl. A61M 16/06; C08K 3/32; C08K 3/40; C08K 9/02; C08K 3/015

(52) U.S. Cl. CPC A61M 16/0666; C08K 3/32; C08K 3/40; C08K 9/02; C08K 3/015; C08K 2003/321; B33Y 10/00

(57) ABSTRACT

A method includes forming an antimicrobial blend including an antimicrobial additive combined with a polymer, and forming a medical device with the antimicrobial blend, wherein a surface of the medical device exhibits antimicrobial properties.

### RELATED PATENT APPLICATIONS

This application claims priority to (a) U.S. Provisional Patent Application No. 63/229,707 filed Aug. 5, 2021 and (b) U.S. Provisional Patent Application No. 63/236,103 filed Aug. 23, 2021, the entire contents of which applications are hereby incorporated by reference for all purposes.

### TECHNICAL FIELD

The present disclosure relates to medical devices, and more particularly to a medical devices having antimicrobial properties and method of forming medical devices having antimicrobial properties.

### BACKGROUND

Medical devices that communicate fluids (gases and/or liquids) to or from a person or otherwise interface with a person, for example tubing, containers, masks, and other devices, frequently become contaminated with bacteria and/or viral matter. Protocols exist for cleaning and sanitizing medical devices, but they are typically time consuming and often overlooked or not performed routinely. This can lead to increased infections, respiratory issues such as pneumonia, and other disease or illness, as well as increasing the required frequency for replacing the contaminated medical devices.

### SUMMARY

One aspect of the present disclosure provides a method including forming an antimicrobial blend including a quantity of an antimicrobial additive combined with a quantity of polymer, and forming a medical device with the antimicrobial blend, wherein a surface of the medical device exhibits antimicrobial properties.

In some embodiments, the medical device comprises a conduit configured to carry a fluid, and forming the medical device with the antimicrobial blend comprises using an extruder to the conduit from the antimicrobial blend, wherein an interior surface of the extruded conduit exhibits antimicrobial properties.

In some embodiments, forming the medical device with the antimicrobial blend comprises using a molding system to perform a molding process to form the medical device from the antimicrobial blend;

In some embodiments, forming the medical device with the antimicrobial blend comprises using an additive manufacturing machine to print the medical device from the antimicrobial blend.

In some embodiments, the antimicrobial additive comprises at least one of (a) silver phosphate glass oxide or (b) copper oxide. In other embodiments, the antimicrobial additive comprises silver, silver oxide, copper, or copper phosphate glass oxide.

In some embodiments, a percentage-by-weight of the antimicrobial additive relative to the polymer is in the range of 0.5% to 10%.

In some embodiments, a percentage-by-weight of the antimicrobial additive relative to the polymer is at least 2.5%. In particular embodiments, a percentage-by-weight of the antimicrobial additive relative to the polymer is in the range of 2.5% to 10%.

In some embodiments, a percentage-by-weight of the antimicrobial additive relative to the polymer is in the range of 3.5% to 5%.

In some embodiments, the polymer comprises a thermoplastic polymer. For example, In some embodiments the polymer comprises polyethylene, polypropylene, polycarbonate, or silicone.

In some embodiments, the antimicrobial additive comprises silver phosphate glass oxide, and the percentage-by-weight of the silver phosphate glass oxide relative to the polymer is in the range of 2.5% to 10%.

Another aspect provides a medical device including an antimicrobial structure comprising a mixture of (a) a polymer and (b) an antimicrobial additive comprising at least one of silver phosphate glass oxide or copper oxide, silver, silver oxide, copper, or copper phosphate glass oxide. A surface of the antimicrobial structure exhibits antimicrobial properties.

In some embodiments, a percentage-by-weight of the antimicrobial additive relative to the polymer is in the range of 0.5% to 10%. In particular embodiments, a percentage-by-weight of the antimicrobial additive relative to the polymer is in the range of 2.5% to 10%. In some embodiments, the surface of the antimicrobial structure provides at least 99.85% reduction (log 2 efficacy) of E. coli bacteria and at least 99.99% reduction (log 4 efficacy) of S. aureus bacteria.

In some embodiments, a percentage-by-weight of the antimicrobial additive relative to the polymer is in the range of 3.5% to 10%. In some embodiments, the surface of the antimicrobial structure provides at least 99.99% reduction (log 4 efficacy) of both E. coli bacteria and S. aureus bacteria.

In some embodiments, the antimicrobial additive comprises silver phosphate glass oxide, and the percentage-by-weight of the silver phosphate glass oxide relative to the polymer is in the range of 3.5% to 10%.

In some embodiments, the antimicrobial structure comprises a conduit for carrying a fluid toward or away from a user.

In some embodiments, the conduit comprises a component of a CPAP system, BiPAP system, or ventilator system.

In some embodiments, the antimicrobial structure comprises a respiratory mask or a component of a respiratory mask.

Another aspect provides a medical device including an antimicrobial structure comprising a mixture of (a) polyethylene, polypropylene, polycarbonate, or silicone, and (b) an antimicrobial additive comprising silver phosphate glass oxide or copper oxide, wherein a percentage-by-weight of the silver phosphate glass oxide relative to the polymer is in the range of 2.5% to 10%, and wherein a surface of the antimicrobial structure provides at least 99.85% reduction (log 2 efficacy) of E. coli bacteria and at least 99.99% reduction (log 4 efficacy) of S. aureus bacteria.

Another aspect provides an antimicrobial medical device formed by a process including forming an antimicrobial blend including a quantity of an antimicrobial additive combined with a quantity of polymer, and forming the medical device with the antimicrobial blend, wherein at least one surface of the medical device exhibits antimicrobial properties.

### BRIEF DESCRIPTION OF THE DRAWINGS

Example aspects of the present disclosure are described below in conjunction with the figures, in which:

FIG. 1 is a flowchart showing an example process for forming an antimicrobial medical device, according to one example embodiment;

FIG. 2 shows an example extruder for extruding the antimicrobial blend to form various types of antimicrobial medical devices;

FIG. 3 is a flowchart showing an example process for forming antimicrobial tubing using the example extruder shown in FIG. 2, according to one example embodiment.

FIG. 4A shows an example length of antimicrobial tubing formed according to the example method shown in FIG. 3;

FIG. 4B shows an example helical ribbed tube including antimicrobial tubing and a helical structural member, formed according to the example method shown in FIG. 3;

FIG. 4C shows an example antimicrobial tubing device including a helical ribbed tube with cuffs attached at both ends;

FIG. 5 shows an example CPAP system including antimicrobial tubing and/or other antimicrobial medical device(s) formed according to any of the techniques as described herein;

FIG. 6 shows an example ventilator system including antimicrobial tubing and/or other antimicrobial medical device(s) formed according to any of the techniques as described herein;

FIG. 7 shows an example humidifier system including antimicrobial tubing and/or other antimicrobial medical device(s) formed according to any of the techniques as described herein;

FIG. 8 shows an example nasal cannula system including antimicrobial tubing and/or other antimicrobial medical device(s) formed according to any of the techniques as described herein;

FIG. 9 shows an example bubble humidifier including antimicrobial medical device(s) formed according to any of the techniques as described herein;

FIG. 10 shows an example CPAP system with an integrated humidifier including antimicrobial medical device(s) formed according to any of the techniques as described herein;

FIG. 11 shows an example aerosol delivery system including antimicrobial tubing and/or other antimicrobial medical device(s) formed according to any of the techniques as described herein;

FIG. 12 shows an example stand-alone nebulizer including antimicrobial tubing and/or other antimicrobial medical device(s) formed according to any of the techniques as described herein;

FIGS. 13A and 13B show an example metered dose and an example MDI spacer including antimicrobial medical device(s) formed according to any of the techniques as described herein;

FIG. 14 shows an example expiration peak flow meter including antimicrobial medical device(s) formed according to any of the techniques as described herein;

FIG. 15 shows an example suction canister including antimicrobial tubing and/or other antimicrobial medical device(s) formed according to any of the techniques as described herein;

FIG. 16 shows an example respiratory mask including antimicrobial medical device(s) formed according to any of the techniques as described herein;

FIG. 17 shows an example nasal pillows respiratory interface including antimicrobial medical device(s) formed according to any of the techniques as described herein;

FIG. 18 shows an example endotracheal tube including antimicrobial medical device(s) formed according to any of the techniques as described herein;

FIG. 19 shows an example laryngeal seal device including antimicrobial medical device(s) formed according to any of the techniques as described herein;

FIGS. 20A and 20B show example breast pump systems including antimicrobial medical device(s) formed according to any of the techniques as described herein; and

FIG. 21 shows an example intravenous (IV) therapy system including antimicrobial tubing and/or other antimicrobial medical device(s) formed according to any of the techniques as described herein.

It should be understood the reference number for any illustrated element that appears in multiple different figures has the same meaning across the multiple figures, and the mention or discussion herein of any illustrated element in the context of any particular figure also applies to each other figure, if any, in which that same illustrated element is shown.

### DETAILED DESCRIPTION

The present disclosure provides medical devices with antimicrobial properties, referred to herein as “antimicrobial medical devices,” and methods of forming medical devices with antimicrobial properties. As used herein, “medical devices” include any tangible systems, devices, instruments, apparatuses, implements, machines, appliances, or implants that can be used for a medical purpose, and any portion, component, or element of the above. Some example medical devices include:

Medical tubing, cannula, or other conduit for transporting fluids (e.g., air, oxygen, other gases, liquids, etc.);

A respiratory mask or other respiratory device interface (e.g., a CPAP mask, BiPAP system, ventilator mask, nasal pillows, etc.), or a component thereof, for example, a flexible face cushion or membrane, a frame, a housing, a forehead pad, headgear, straps, an elbow, a swivel, or other connection element.

Any other component of a respiratory system or device, for example a component of a continuous positive airway pressure (CPAP) system, bilevel positive airway pressure (BiPAP) system, ventilator, oxygen concentrator, humidifier, nebulizer, etc.

Any component of a metered dose inhaler (MDI), expiration peak flow meter, suction flow chamber device, breast pump system, intravenous IV therapy system, etc.

Some embodiments provide an antimicrobial medical device formed from an antimicrobial blend including an antimicrobial additive mixed with or otherwise added to a polymer (e.g., polyethylene, polypropylene, polycarbonate, silicone, etc.), using any suitable fabrication process to form the relevant medical device.

In some embodiments, the antimicrobial additive comprises silver phosphate glass oxide. In other embodiments, the antimicrobial additive comprises copper oxide. In still other embodiments, the antimicrobial additive comprises silver, silver oxide, copper, or copper phosphate glass oxide.

In some embodiments, the polymer to which the antimicrobial additive is added may comprise any one or more of the following:
(a) a thermoplastic polymer, for example polyethylene (PE), chlorinated polyethylene (CPE), polypropylene (PP), polycarbonate (PC), polyamide, polyolefin, thermoplastic elastomer (TPE), e.g., polyolefin thermoplastic elastomer (P-TPE), thermoplastic olefin (TPO), ethylene propylene diene monomer (EPDM), ethylene-vinyl acetate (EVA), thermoplastic polyurethane (TPU), styrene-ethylene-butylene-styrene (SEBS), or a co-polymer combination,
(b) ethylene polysulfide (ET) or ethylene-propylene copolymer (EPM),
(c) silicone (e.g., SILASTIC′ silicone rubber by Dow Corning) or other organopolysiloxane (SI) or polyorganosiloxane,
(d) polybutadiene (rubber), polyisoprene (synthetic rubber), or vulcanized rubber,
(e) a styrene-butadiene copolymer, styrene-butadiene, styrene-butadiene rubber (SBR), styrene butadiene styrene (SBS), styrene isoprene styrene (SIS), styrene ethylene/propylene styrene (SEPS), styrene ethylene/butylene styrene (SEBS), styrene ethylene/ethylene propylene styrene (SEEPS), or styrenic block copolymer (SBC), for example, K-Resin® SBC by Entec Polymers, Orlando, Fla.,
(f) a gelatinous elastomeric composition comprising a mixture of a thermoplastic elastomer and an oil (or plasticizer), for example a mixture of a styrenic block copolymer and anoil,
(g) other gel, or
(h) other polymer suitable for mixing with an antimicrobial additive.

In some embodiments, exposed surfaces of the antimicrobial medical device may exhibit antimicrobial properties, for example providing an antimicrobial efficacy of at least 99% (log 2 efficacy), 99.9% (log 3 efficacy), or 99.99% (log 4 efficacy) with respect to various bacteria, e.g., E. coli and/or S. aureus bacteria, according to ISO 22196 testing standards, as discussed in more detail below.

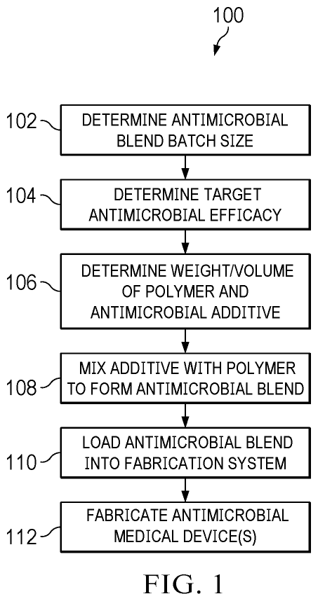

FIG. 1 is a flowchart showing an example process 100 for forming an antimicrobial medical device according to one example embodiment. The antimicrobial medical device may comprise any of the example medical devices disclosed in the present description or drawings, or any other type of medical device. For example, in some embodiments the antimicrobial medical device may comprise antimicrobial flexible tubing, e.g., for use in a CPAP system, BiPAP system, ventilator system, or other respiratory system or other medical device. As another example the antimicrobial medical device may comprise a rigid or semi-rigid medical device, e.g., rigid or semi-rigid tubing, a component of a mask, a canister or reservoir, or a connection element. As another example the antimicrobial medical device may comprise a flexible cushion or membrane for a mask, or a flexible bladder or diaphragm.

At 102, a batch size of an antimicrobial blend for forming the relevant antimicrobial medical device. The batch size may represent a weight or volume of the antimicrobial blend sufficient for forming the antimicrobial medical device or a specified quantity of the antimicrobial medical device. For example, a 50 pound batch size may be selected for producing a specified length of medical tubing.

At 104, a target antimicrobial efficacy may be selected or determined for the antimicrobial medical device(s) being produced. For example, the target antimicrobial efficacy may be defined by the standards set forth in ISO 22196 or other standard, e.g., an efficacy of 90%, 95%, 98%, 99% (log 2), 99.9% (log 3), 99.99% (log 4), or 99.999% (log 5), or other value, with respect to particular bacteria (e.g., E. coli and/or S. aureus bacteria).

At 106, a quantity, e.g., a weight or volume, of each component of the antimicrobial blend may be determined based on the selected batch size and target antimicrobial efficacy. For example, for an antimicrobial blend including a polymer (e.g., polyethylene, polypropylene, polycarbonate, or silicone) and an antimicrobial additive (e.g., silver phosphate glass oxide or copper oxide), the weight and/or volume of the polymer and the antimicrobial additive may be calculated. The polymer may be provided in the form of a resin, powder, pellets, gel, oil, liquid, or other form suitable for combination with an antimicrobial additive to form an antimicrobial blend that may be processed by a fabrication system suitable to fabricate the antimicrobial medical device, for example an extruder, an injection molding system, an injection blow molding system, an extrusion blow molding system, a vacuum forming system, or an additive printer (e.g., a 2.5D or 3.5D printer).

In some embodiments, a percentage-by-weight (wt %) of the antimicrobial additive relative to the polymer resin, also referred to herein as the “blended ratio,” may be selected or determined for providing the target antimicrobial efficacy, e.g., based on predefined reference data. For example, the inventor has discovered relationships between the antimicrobial additive wt % in the antimicrobial blend (blended ratio) and the antimicrobial efficacy of an antimicrobial medical device produced (e.g., extruded, molded, printed, etc.) from the antimicrobial blend, for a particular set of process parameters.

For example, the inventor has found the following relationship between antimicrobial additive wt % (blended ratio) and antimicrobial efficacy, for example medical tubing produced according to one embodiment:

TABLE 1

| Antimicrobial | ISO 22196 efficacy | |
|---|---|---|
| additive wt % | E. coli | S. aureus |
| 2.5% | ≥99.99% (log 4) | ≥99.85% (log 2) |
| 3.5% | ≥99.99% (log 4) | ≥99.99% (log 4) |
| 5% | ≥99.99% (log 4) | ≥99.99% (log 4) |

It should be understood that the values in Table 1 are experimental data points for a particular batch of tubing. In other embodiments, the antimicrobial efficacy (with respect to E. coli and/or S. aureus) of the relevant antimicrobial medical device produced in accordance with the present disclosure may be better or worse, as a function of various process parameters or variables, the particular fabrication process, and the type of component being produced. For example, in some embodiments an antimicrobial medical device containing less than 3.5% (wt %) antimicrobial additive (e.g., silver phosphate glass oxide) may provide ≥99.99% (log 4) efficacy for both E. coli and/or S. aureus.

In some embodiments, for an antimicrobial blend including silver phosphate glass oxide (antimicrobial additive) mixed with a thermoplastic polymer, e.g., polyethylene, a blended ratio in the range of 0.5-10% (or in some embodiments 1-10%) is used, which may provide desired antimicrobial properties. In particular embodiments, a blended ratio of at least 2.5%, for example in the range of 2.5-10%, is used, which may provide at least 99.85% (log 2) reduction of E. coli bacteria and at least 99.99% (log 4) reduction of S. aureus bacteria, according to the example test data shown in Table 1.

In other embodiments, a blended ratio of at least 3.5% is used, which may provide at least 99.99% (log 4) reduction of both E. coli bacteria and S. aureus bacteria, according to the example test data shown in Table 1. In particular embodiments, a blended ratio in the range of 3.5%-5% is used, which may yield at least 99.99% (log 4) reduction of both E. coli bacteria and S. aureus bacteria, with a limited quantity of antimicrobial additive.

Thus, based on identified relationships between antimicrobial additive wt % (blended ratio) and antimicrobial efficacy, the weight or volume of each component of the antimicrobial blend may be determined based on the selected batch size and a selected wt % corresponding with a target antimicrobial efficacy.

In some embodiments, the antimicrobial additive may comprise any of the example additives listed above (e.g., silver phosphate glass oxide, copper oxide, or other suitable antimicrobial compound. In some embodiments, the antimicrobial additive comprises silver, silver oxide, copper, or copper phosphate glass oxide), and the polymer may comprise any of the example polymers listed above (e.g., polyethylene, polypropylene, polycarbonate, silicone, etc.)

In some embodiments, the antimicrobial blend may include additional components (in addition to the polymer and antimicrobial additive), for example any one or more of the following: reinforcing fibers (e.g., carbon, Kevlar, or glass fibers), oil or plasticizer (e.g., anoil, mineral oil, or silicone (dimethyl siloxane) oil), a UV-stabilizer, a heat-stabilizer, antiviral agents, antifungus agents, antioxidants, pigments, glitters, dyes, or combinations thereof.

At 108, the antimicrobial additive may be mixed with the polymer (e.g., in the form of a polymer resin), e.g., using gravimetric, optometric, or volumetric dosing systems, or by manually weighing components and tumble mixing batches. In some embodiments, batch mixing may avoid potential problems or complications associated with certain dosing systems.

For example, a 50 pound batch of PE resin may be weighed and prepped for the antimicrobial additive. The weight of antimicrobial additive to be added may be calculated as described above at 106. The antimicrobial additive may then be weighed and prepped for mixing. The antimicrobial additive batch and PE resin batch may be added to a multi-axis tumble mixer and tumble mixed, e.g., for 2-10 minutes, to provide a thorough dispersion of the antimicrobial additive within the PE resin. This antimicrobial additive/PE dispersion, referred to herein as the antimicrobial blend, may be used for producing the relevant antimicrobial medical device(s).

At 110, the antimicrobial blend is loaded into a fabrication system suitable for producing the relevant antimicrobial medical device(s). For example, the antimicrobial blend may be loaded into a hopper or other inlet of an extruder, an injection molding system, an injection blow molding system, an extrusion blow molding system, a vacuum forming system, or an additive printer (e.g., a 2.5D or 3.5D printer).

At 112, the relevant fabrication system is operated to produce the antimicrobial medical device(s) from the antimicrobial blend. For example, the antimicrobial medical device may be extruded, molded, vacuum-formed, or printed using any fabrication systems disclosed herein or other suitable fabrication system.

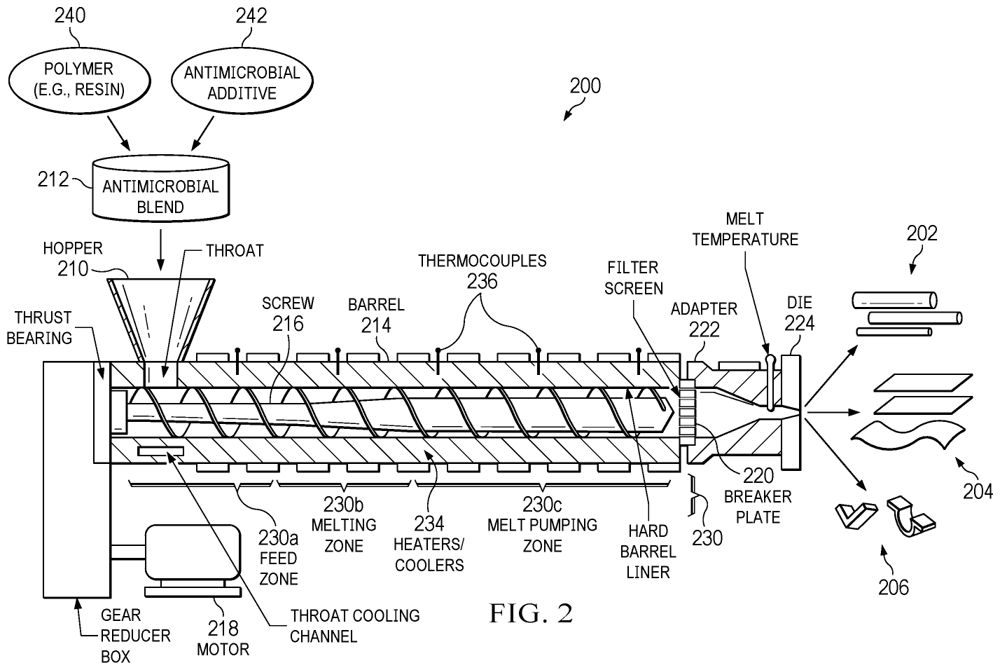

FIG. 2 shows an example extruder 200 for extruding the antimicrobial blend to form various types of antimicrobial medical devices. For example, extruder 200 may be used to extrude tubing 202, sheets or films 204, and/or other three-dimensional structures 206. Extruder 200 may include a hopper 210 for receiving an antimicrobial blend 212 into the interior of a barrel 214, wherein a screw 216 driven by a motor 218 drives the antimicrobial blend 212 through various extruder zones 230 and through a downstream breaker plate 220, adapter 222, and die 224 that creates the desired output structure, e.g., tubing 202, a sheet or film 204, or other three-dimensional structure 206. In this example, the extruder zones 230 may include a feed zone 230a, a melting zone 230b, and a melt pumping zone 230c. Heating and cooling devices 234 and thermocouples or other temperature sensors 234 may be positioned relative to respective extruder zone 230a-230c for controlling the temperature of the antimicrobial blend 212 as it progresses through the barrel 214. Operational parameters of each extruder zone 230a-230c may be set and controlled for a specified processing of the antimicrobial blend 212. For example, motor 218 may control a speed of the screw 216 (screw speed), and heating and cooling devices 234 may control a temperature in each respective extruder zone 230a-230c (or a temperature of the antimicrobial blend 212 in each respective extruder zone 230a-230c).

In other embodiments, antimicrobial tubing and/or other types of antimicrobial medical devices may be formed by pultrusion using a pultrusion system that pulls the antimicrobial blend through a die, as opposed to extrusion in which the antimicrobial blend is pushed through a die. For example, in some embodiments, pultrusion may be used to form antimicrobial medical devices from an antimicrobial blend that includes reinforcing fibers, for example carbon, Kevlar, or glass fibers, mixed with the polymer and antimicrobial additive.

In other embodiments, antimicrobial medical devices may be formed by injection molding using an injection molding system. For example, in some embodiments, an injection molding system may be used to form various rigid or semi-rigid medical devices, for example various mask component of a mask, canisters or reservoirs, and connection elements, from an antimicrobial blend including an antimicrobial additive mixed with a suitable polymer (for example polycarbonate, polyethylene, polypropylene, polyurethane, or a styrenic block copolymer, e.g., K-Resin® SBC). As another example an injection molding system may be used to form various flexible medical devices, for example a flexible shell and/or face cushion or membrane for a mask, or a flexible bladder, gasket, or diaphragm, from an antimicrobial blend including an antimicrobial additive mixed with a suitable polymer (for example silicone (e.g., SILASTIC′), polyethylene, polypropylene, thermoplastic polyurethane, or an elastomeric gel, e.g., a gelatinous elastomeric composition comprising a mixture of a thermoplastic elastomer and an oil or plasticizer).

In some embodiments, an injection molding system may be used to form medical devices having different rigidity/flexibility from the same antimicrobial blend. For example, multiple medical device components having different rigidity/flexibility may be injection molded from the same antimicrobial blend, e.g., by varying the thickness of the respective injection molded components, or by varying a material density of the respective injection molded components using a gas-assisted injection molding system, wherein the respective material density provides a corresponding rigidity/flexibility of the respective injection molded components. For example, a rigid or semi-rigid frame and a flexible cushion (or membrane) for a mask may both be injection molded from the same antimicrobial blend (e.g., comprising an antimicrobial additive mixed with polypropylene, polyethylene, or silicone) by varying a thickness of the respective injection molded components, or by varying a material density of the respective injection molded components.

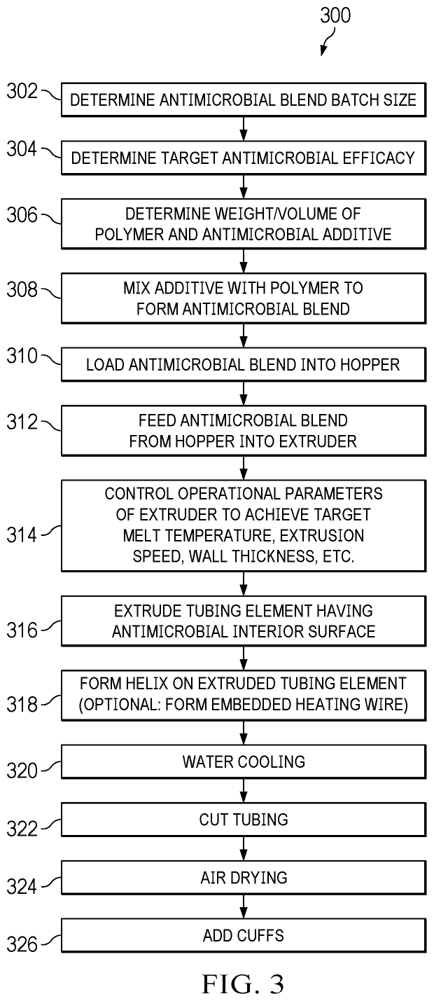

FIG. 3 is a flowchart showing an example process 300 for forming an example antimicrobial medical device, in particular antimicrobial tubing, using the example extruder 200 shown in FIG. 2, according to one example embodiment.

At 302, a batch size of an antimicrobial blend 212 for extruding antimicrobial tubing is determined, e.g., for forming air/oxygen delivery tubes for CPAP machines, BiPAP machines, ventilators, or other type of respiratory system or other medical device. The batch size may represent a weight or volume of the antimicrobial blend 212 sufficient for extruding a target length of tubing (e.g., to cut into a target number of tubing products) with target specifications (e.g., length, wall thickness, etc.). For example, a 50 pound batch size may be selected for producing a target number of CPAP inhalation tubes.

At 304, a target antimicrobial efficacy may be selected or determined for the tubes to be produced. For example, the target antimicrobial efficacy may be defined by the standards set forth in ISO 22196 or other standard, e.g., an efficacy of 90%, 95%, 98%, 99% (log 2), 99.9% (log 3), 99.99% (log 4), or 99.999% (log 5), or other value, with respect to particular bacteria (e.g., E. coli and/or S. aureus bacteria).

At 306, a quantity, e.g., a weight or volume, of each component of the antimicrobial blend 212, may be determined based on the selected batch size and target antimicrobial efficacy. For example, for an antimicrobial blend 212 including a polymer 240 (e.g., polyethylene or polypropylene) and an antimicrobial additive 242 (e.g., silver phosphate glass oxide or copper oxide), the weight and/or volume of the polymer 240 and the antimicrobial additive 242 may be calculated. The polymer 240 may be provided in the form or a resin or other form suitable for combination with an antimicrobial additive 242 to form an antimicrobial blend 212 that may be processed by the extruder 200.

In some embodiments, a percentage-by-weight (wt %) of the antimicrobial additive relative to the polymer resin (blended ratio) may be selected or determined for providing the target antimicrobial efficacy, e.g., based on predefined reference data. For example, as discussed above with reference to Table 1, the inventor has discovered relationships between the antimicrobial additive wt % in the antimicrobial blend (blended ratio) and the antimicrobial efficacy of tubing extruded from the antimicrobial blend, for a particular set of process parameters.

For example, in some embodiments, for an antimicrobial blend including silver phosphate glass oxide (antimicrobial additive) mixed with a thermoplastic polymer, e.g., polyethylene, a blended ratio in the range of 0.5-10% (or in some embodiments 1-10%) is used, which may provide desired antimicrobial properties. In particular embodiments, a blended ratio of at least 2.5%, for example in the range of 2.5-10%, is used, which may provide at least 99.85% (log 2) reduction of E. coli bacteria and at least 99.99% (log 4) reduction of S. aureus bacteria, according to the example test data shown above in Table 1.

In other embodiments, a blended ratio of at least 3.5% is used, which may provide at least 99.99% (log 4) reduction of both E. coli bacteria and S. aureus bacteria, according to the example test data shown in Table 1 above. In particular embodiments, a blended ratio in the range of 3.5%-5% is used, which may yield at least 99.99% (log 4) reduction of both E. coli bacteria and S. aureus bacteria, with a limited quantity of antimicrobial additive.

Thus, based on identified relationships between antimicrobial additive wt % (blended ratio) and antimicrobial efficacy, the weight or volume of each component 240, 242 of the antimicrobial blend 212 may be determined based on the selected batch size and a selected wt % corresponding with a target antimicrobial efficacy.

In some embodiments, the polymer 240 may comprise a thermoplastic polymer, for example polyethylene (PE), polypropylene (PP), polyolefin, thermoplastic elastomer (TPE), e.g., polyolefin thermoplastic elastomer (P-TPE), thermoplastic olefin (TPO), ethylene propylene diene monomer (EPDM), ethylene-vinyl acetate (EVA), thermoplastic polyurethane (TPU), styrene-ethylene-butylene-styrene (SEBS), or a co-polymer combination.

In some embodiments, the antimicrobial additive 242 may comprise silver phosphate glass oxide, copper oxide, or other suitable antimicrobial compound. In some embodiments, the antimicrobial additive 242 comprises silver, silver oxide, copper, or copper phosphate glass oxide.

At 308, the antimicrobial additive 242 may be mixed with the polymer 240 (e.g., polymer resin), for example using gravimetric, optometric, or volumetric dosing systems, or by manually weighing components and tumble mixing batches. In some embodiments, batch mixing may avoid potential problems or complications associated with certain dosing systems.

For example, a 50 pound batch of PE resin 240 may be weighed and prepped for the antimicrobial additive 242. The weight of antimicrobial additive 242 to be added may be calculated as described above at 306. The antimicrobial additive 242 may then be weighed and prepped for mixing. The antimicrobial additive batch 242 and PE resin batch 240 may be added to a multi-axis tumble mixer and tumble mixed, e.g., for 2-10 minutes, to provide a thorough dispersion of the antimicrobial additive 242 within the PE resin 240, thereby forming the antimicrobial blend 212.

At 310, the antimicrobial blend 212 may be loaded into the extruder hopper 210. At 312, the antimicrobial blend 212 may be fed from the hopper 202 into the barrel 214.

At 314, various operational parameters of the extruder 200 may be set and/or controlled, and the antimicrobial blend 212 may be processed at 316 to extrude a length of tubing 202. For example, temperatures in respective extruder zones 230a-230c may be set in the range of 250-650° F. while processing the antimicrobial blend 212. In some embodiments, one or more extruder zones 230a-230c may be maintained in the range of 300-500° F. to provide desired results. In particular embodiments, one or more extruder zones 230a-230c may be maintained in the range of 400-475° F. may provide consistent results of desired melt temperature, back pressure and load on the screw motor 218.

As another example, the screw motor 218 may maintain the screw speed of screw 216 in the range of 10-75 rpm, for example in the range of 20-40 rpm. In some embodiments, a screw speed in the range of 19-25 rpm may provide consistent results and extruded tubing size.

As another example, the antimicrobial blend 212 may be processed with screen pack configurations of 20-150 mesh screen combinations. Some embodiments use a minimum of 20 mesh screens. In one embodiment a screen pack of 30, 60, 60, 100 (listed in order as inserted into breaker plate) may yield desirable or optimum mixing and back pressure. Melt temperature may be measured upstream of the breaker plate 220.

In some embodiments the antimicrobial blend 212 may be processed at a melt temperature in the range of 275-500° F., for example in the range of 300-400° F. for desired processing performance. In one embodiment the antimicrobial blend 212 may be processed at a melt temperature in the range of 330-340° F. for desirable or optimum processing performance while limiting thermal exposure of the antimicrobial additive 242.

In some embodiments, the combination of extruder zone temperature, screw speed, breaker plate screen pack, melt temperature and back pressure parameters disclosed above provide thorough mixing and dispersion of the antimicrobial additive 242 without excess thermal degradation of the antimicrobial additive 242 or polymer 240 (e.g., PE resin).

In some embodiments the extruder 200 is controlled to extrude a tubing element 202 having an extrusion profile (wall thickness) in the range of 0.002″ to 0.012.″ In a particular embodiment, the extruder 200 is controlled to extrude a tubing element 202 having an extrusion profile (wall thickness) in the range of 0.003″ to 0.007″.

At 318, an optional step, a structural member may be added to the extruded tubing element 202, e.g., to increase structural integrity or provide a desired rigidity or flexibility of the tubing element 202. For example, a semi-rigid element (e.g., more rigid than the extruded tubing element 202) may be wrapped around the outside of the extruded tubing element 202 in a helical fashion to form a continuous overlapping tube. In some embodiments, a semi-rigid elongated member is extruded, e.g., from a polypropylene resin, and wrapped helically around the outside of the antimicrobial tubing element 202 and bonded to the tubing element 202 to form a helical ribbed tube, which may provide increased crush resistance and strength to the tube. In some embodiments, the structural member may also be formed as an antimicrobial medical device, e.g., by forming and extruding an antimicrobial blend including an antimicrobial additive (e.g., any of the example antimicrobial additives disclosed herein) added to a polypropylene resin.

In addition, in some embodiments conductive wiring may (optionally) be arranged between the helical structural member and the underlying tubing element 202, which may be connected to heating and temperature feedback circuitry, for example, via connectors formed in a cuff added at step 326 discussed below. For example, three strands of 29 AWG coated copper wire may be inserted under the helical structural member encapsulated into the tubing to be later connected into heating and temperature feedback circuits via connectors in a cuff formed at one end of the tube.

At 320, the tubing may be arranged on a cooling tray and sprayed with or submerged in water or other liquid in the range of 40-80° F., to cool and solidify the tubing product. At 322, the tubing may be cut to size, e.g., to form a number of discrete lengths of tubing. At 324, the tubing products may be transferred to a high velocity air dryer to strip any remaining water droplets from the tubes. At 326, a cuff (e.g., configured for connection to other components of a respiratory assistance device) may be attached to one or both ends of each length of tubing.

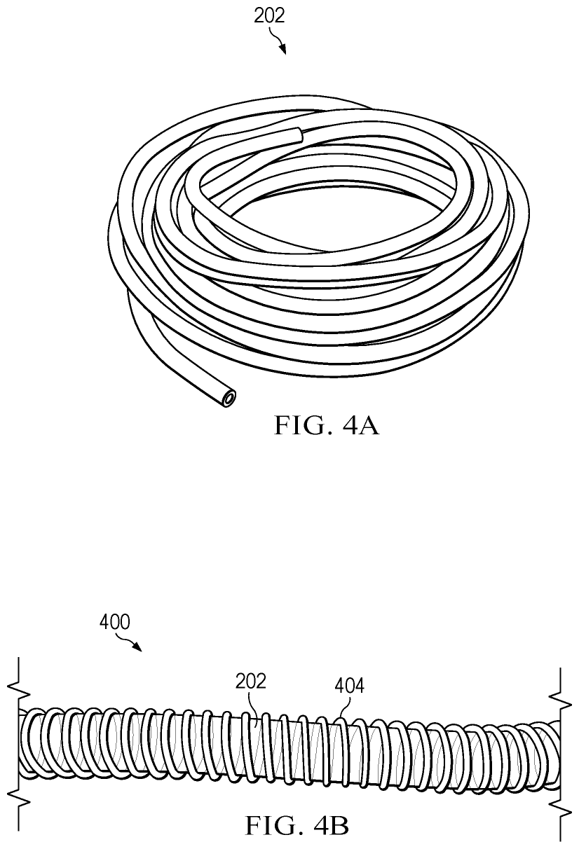

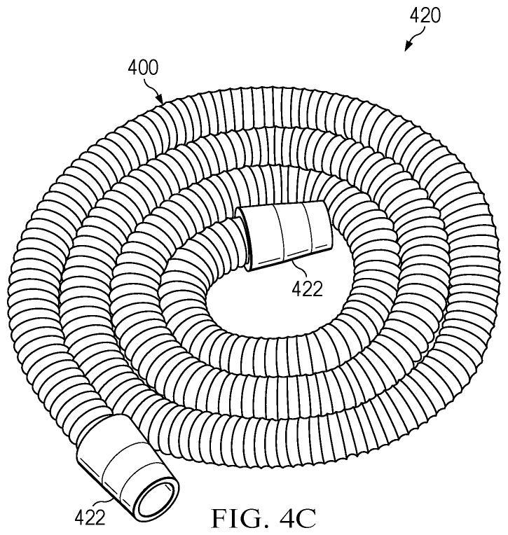

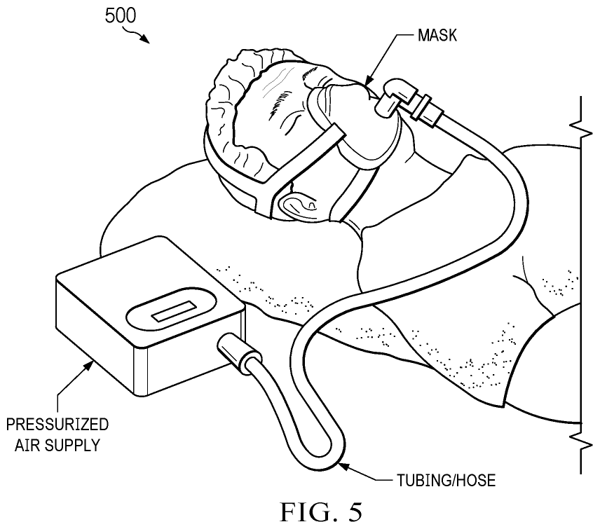

FIG. 4A shows an example length of antimicrobial tubing 202 formed according to the example method 300 discussed above, e.g., without the optional structural member described above at 318. Antimicrobial tubing 202 may be used for a nasal cannula or used with various other medical systems and devices.

FIG. 4B shows an example helical ribbed tube 400 formed according to the example method 300 discussed above, e.g., including the optional structural member described above at 318. The helical ribbed tube 400 includes the antimicrobial tubing element 202 and a semi-rigid structural member 404 wrapped helically around the outside of the antimicrobial tubing element 202, which may be formed as described above regarding step 318.

FIG. 4C shows an example antimicrobial tubing device 420 including the helical ribbed tube 400 with cuffs 422 attached at both ends as described above.

Table 2 shows example lab test results for an interior surface of an antimicrobial tubing element 202 formed as disclosed above, and using an antimicrobial blend of polyethylene (polymer) and 2.5% (wt %) silver phosphate glass oxide (antimicrobial additive), according to one example embodiment. As shown, the antimicrobial tubing element sample exhibited a reduction of ≥99.99% (≥log 4) of Escherichia coli (E. coli) bacteria and a reduction of ≥99.85% (≥log 2) of staphylococcal (S. aureus) bacteria, as compared with a reference tubing sample formed from polyethylene but not including the antimicrobial additive (silver phosphate glass oxide).

TABLE 2
Example test results for tubing including 2.5% (wt %) silver phosphate glass oxide

| | Wt % | | Contact Time | | Reduction (Initial) | |
|---|---|---|---|---|---|---|
| Sample | of AA | Species | 0 hrs | 24 hrs | Log 10 | % |
| Tubing without antimicrobial | N/A | E. coli | 1.2E+04 | 5.3E+05 | | |
| additive (AA) | | | | | | |
| Tubing including AA | 2.5% | E. coli | 1.2E+04 | <25.00 | ≥4.33 | ≥99.99% |
| (silver phosphate glass oxide) | | | | | | |
| Tubing without AA | N/A | S. aureus | 1.1E+04 | 1.6E+04 | | |
| Tubing including AA | 2.5% | S. aureus | 1.1E+04 | <25.00 | ≥2.81 | ≥99.85% |
| (silver phosphate glass oxide) | | | | | | |

Similarly, Table 3 shows example lab test results for an interior surface of an antimicrobial tubing element formed as disclosed above, using an antimicrobial blend of polyethylene (polymer) and 3.5% (wt %) silver phosphate glass oxide (antimicrobial additive), according to one example embodiment. As shown, the antimicrobial tubing element sample exhibited a reduction of ≥99.99% (≥log 4) of both E. coli and S. aureus bacteria, as compared with a reference sample formed from polyethylene but not including the antimicrobial additive (silver phosphate glass oxide).

TABLE 3
Example test results for tubing including 3.5% (wt %) silver phosphate glass oxide

| | Wt % | | Contact Time | | Reduction (Initial) | |
|---|---|---|---|---|---|---|
| Sample | of AA | Species | 0 hrs | 24 hrs | Log 10 | % |
| Tubing without antimicrobial | N/A | E. coli | 1.1E+04 | 3.4E+05 | | |
| additive (AA) | | | | | | |
| Tubing including AA | 3.5% | E. coli | 1.1E+04 | <1.00 | ≥4.02 | ≥99.99% |
| (silver phosphate glass oxide) | | | | | | |
| Tubing without AA | N/A | S. aureus | 2.1E+04 | 1.4E+04 | | |
| Tubing including AA | 3.5% | S. aureus | 2.1E+04 | <1.00 | ≥4.32 | ≥99.99% |
| (silver phosphate glass oxide) | | | | | | |

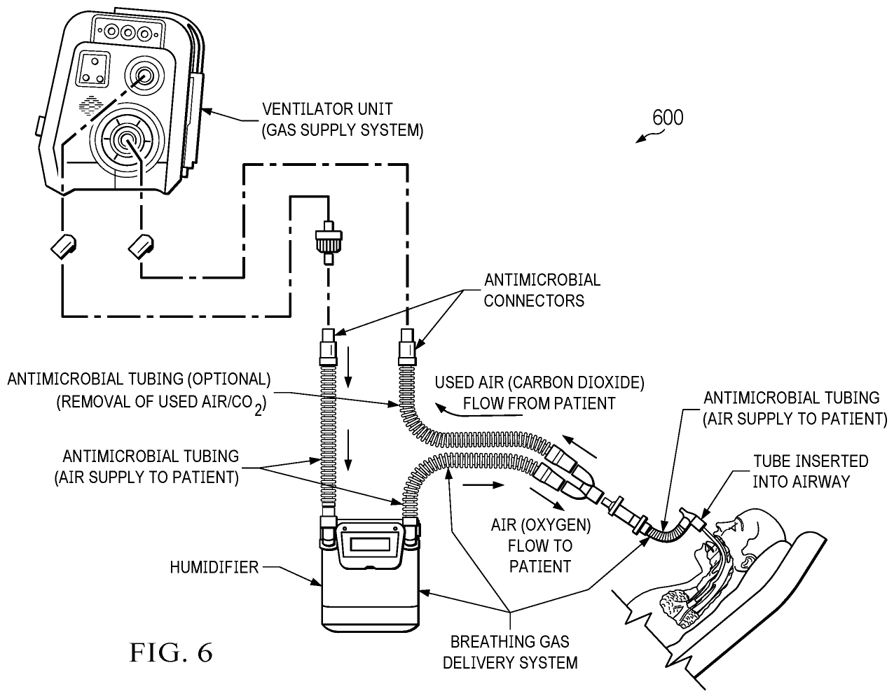

FIGS. 5-21 illustrate example systems and apparatuses including one or more antimicrobial medical devices formed according to any of the techniques disclosed herein. It should be understood that any other polymer-based or polymer-including component of any of the illustrated systems and apparatuses may be formed as an antimicrobial medical device according to the techniques disclosed herein.

FIG. 5 shows an example CPAP system 500 including antimicrobial tubing formed according to any of the techniques as described herein, connected between a pressurized air supply and a face mask, for delivering pressurized air to a person. In some embodiments, the antimicrobial tubing may comprise helical ribbed tube 400.

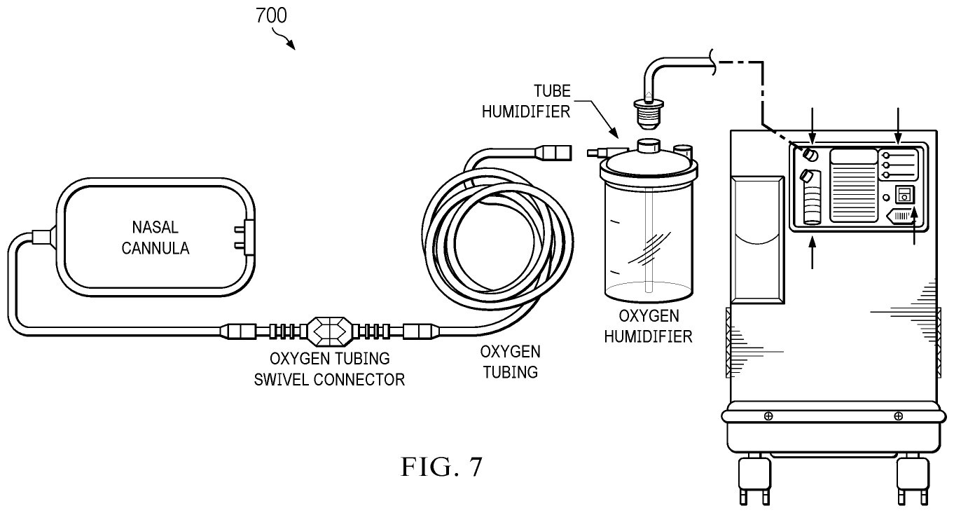

FIG. 6 shows an example ventilator system 600 including antimicrobial tubing formed according to any of the techniques as described herein. As shown, antimicrobial tubing may be used for each section along the supply line for supplying breathing air to the patient, and may also (optionally) be used for each section along the removal line for removing used air (CO₂) from the patient. Other components of the ventilator system 600, e.g., connectors, a humidifier reservoir, etc. may also be formed as antimicrobial devices according to any of the techniques as described herein.

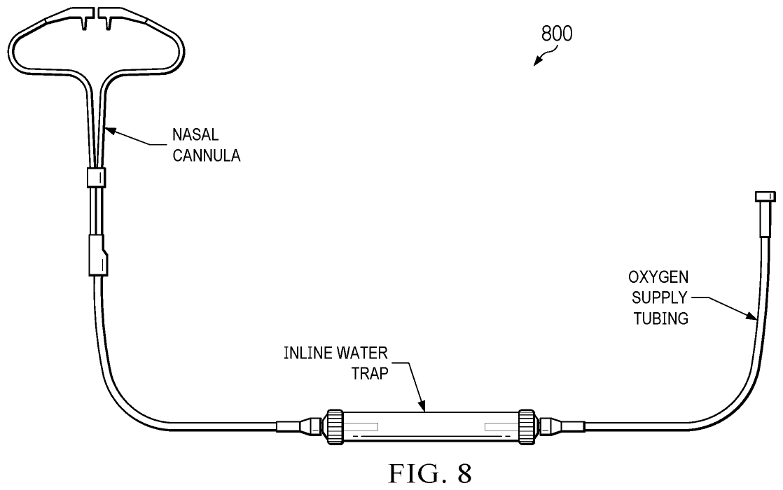

FIG. 7 shows an example humidifier system 700 configured for connection to a ventilator or other respiratory assistance system according to one embodiment. The humidifier system 700 may include one or more antimicrobial medical devices, for example a humidifier bottle, bottle lid, connector, oxygen tubing, a tubing swivel connector, and/or a nasal cannula or a component of a nasal cannula.

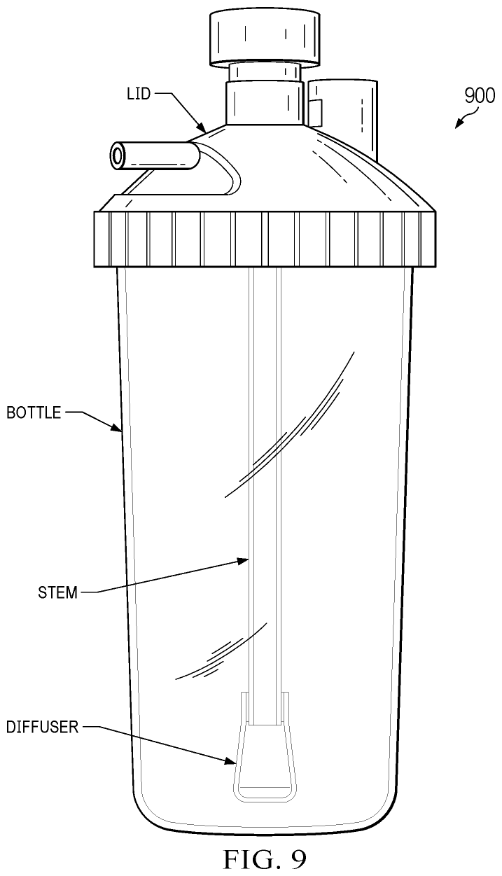

FIG. 8 shows an example nasal cannula system 800 according to one embodiment. The nasal cannula system 800 may include one or more antimicrobial medical devices, for example a cannula, an inline water trap, and/or oxygen supply tubing.

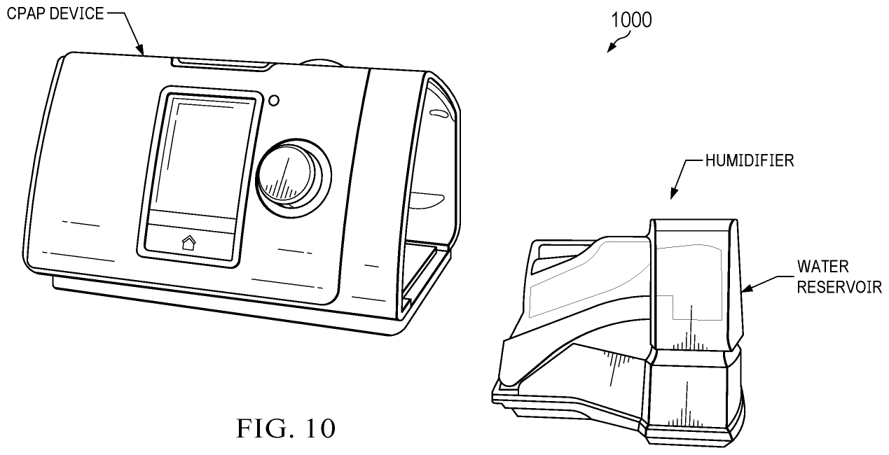

FIG. 9 shows an example bubble humidifier 900 according to one embodiment. The bubble humidifier 900 may include one or more antimicrobial medical devices, for example cannula, a cup or bottle, a lid configured to be secured on the cup or bottle, a stem configured to extend downward from the lid, and/or a diffuser configured for use at a distal end of the stem.

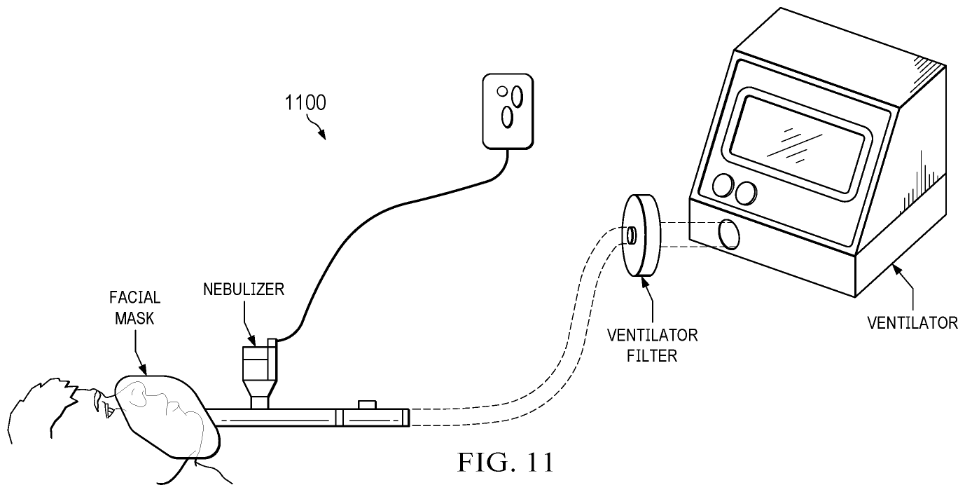

FIG. 10 shows an example CPAP system 1000 including a CPAP device with an integrated humidifier according to one embodiment. The integrated humidifier may include one or more antimicrobial medical device, for example a plastic water reservoir.

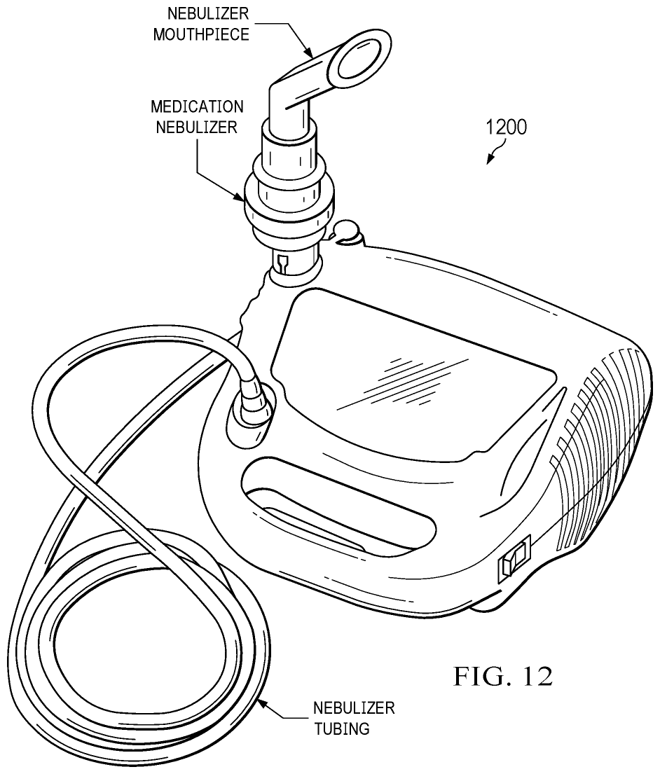

FIG. 11 shows an example aerosol delivery system 1110 including a nebulizer according to one embodiment. The aerosol delivery system may include one or more antimicrobial medical device, for example a nebulizer body, a filter housing or other filter component, and/or a face mask.

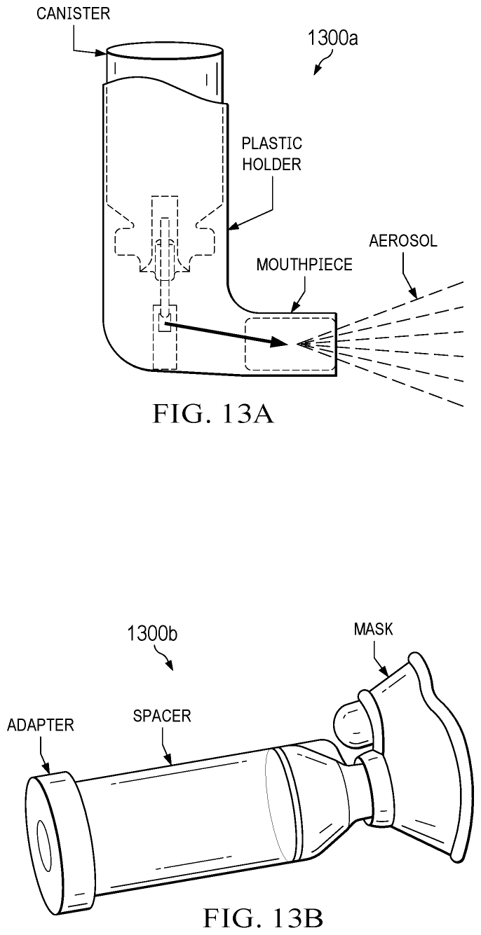

FIG. 12 shows an example stand-alone nebulizer 1200 according to one embodiment. The nebulizer may include one or more antimicrobial medical device, for example a mouthpiece, nebulizer tubing, and/or a face mask.

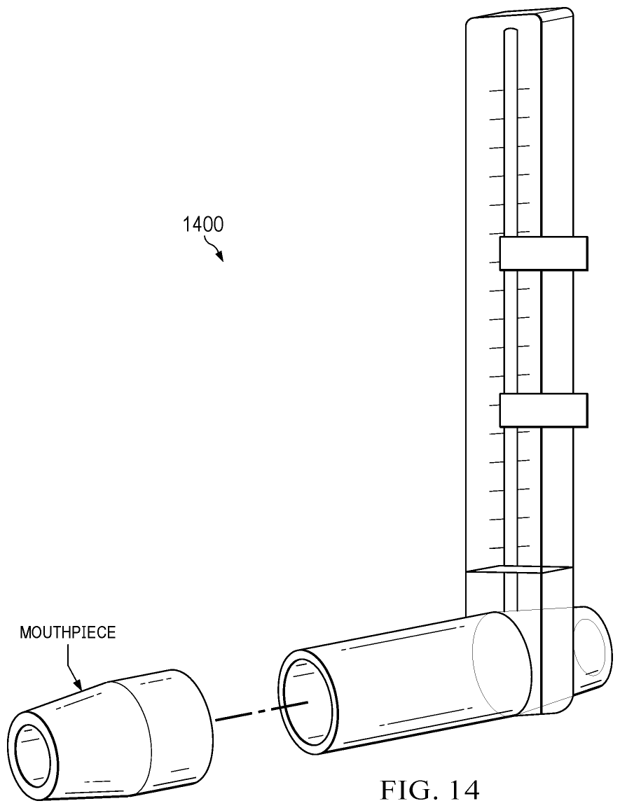

FIG. 13A shows an example metered dose inhaler (MDI) device 1300a according to one embodiment. The MDI device 1300a may include one or more antimicrobial medical device, for example a plastic canister holder, a metering valve, and/or a mouthpiece. FIG. 13B shows an example MDI spacer 1300b according to one embodiment. The MDI spacer 1300b may include one or more antimicrobial medical device, for example an MDI spacer adapter, an MDI spacer body, and/or an MDI spacer mask.

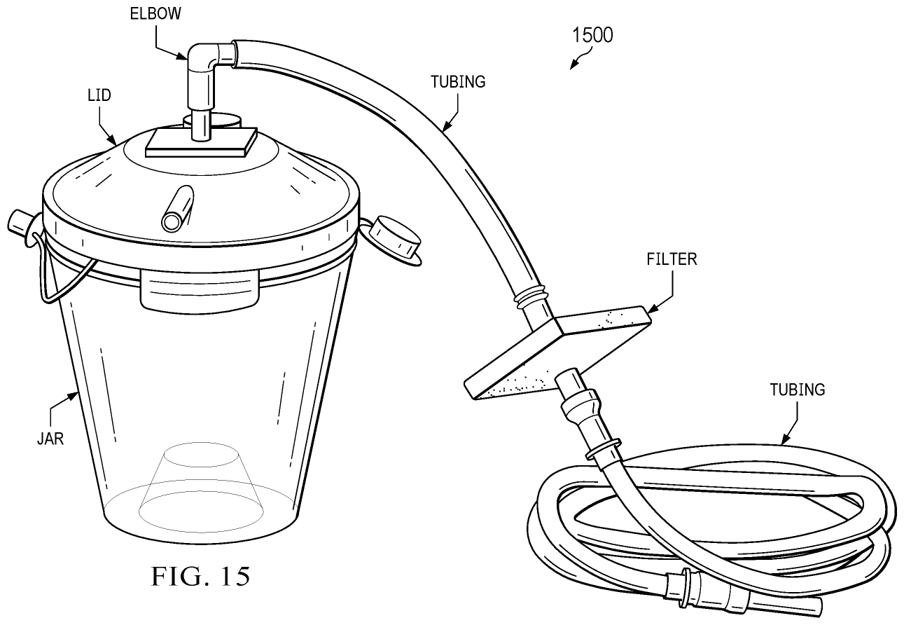

FIG. 14 shows an example expiration peak flow meter 1400 according to one embodiment. The expiration peak flow meter 1400 may include one or more antimicrobial medical device, for example a peak flow meter body and/or a mouthpiece.

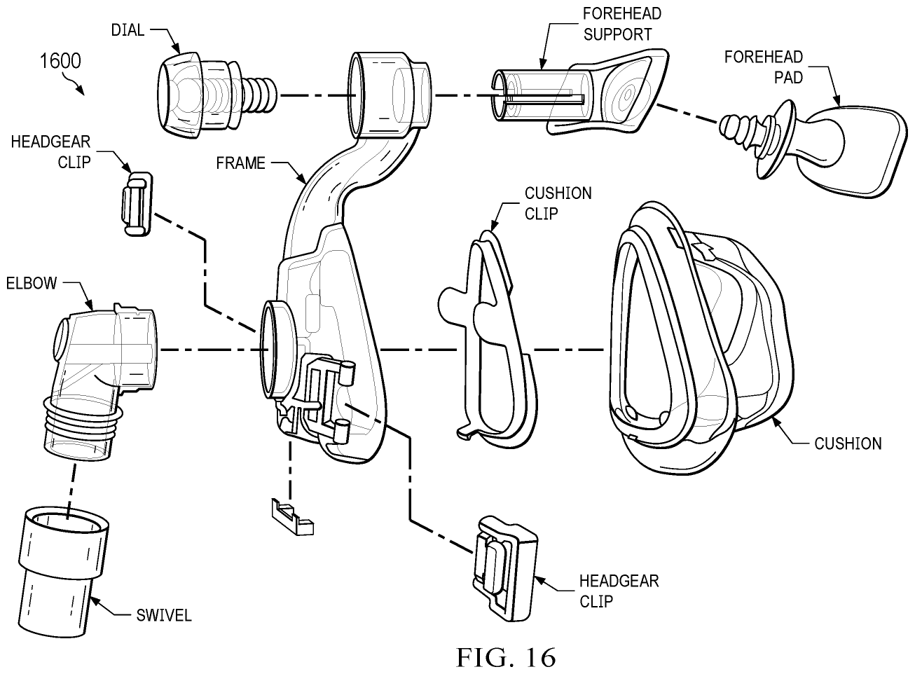

FIG. 15 shows an example suction canister 1500 with antimicrobial tubing, according to one embodiment. The suction canister 1500 with tubing may include one or more antimicrobial medical device, for example a jar, a lid, a vacuum inlet, a filter, and/or antimicrobial tubing.

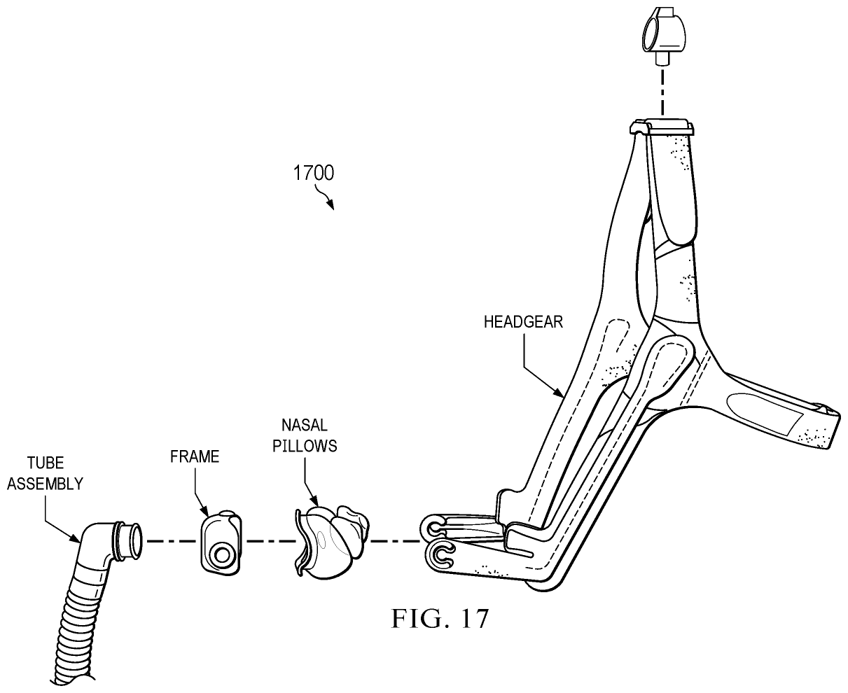

FIG. 16 shows an example respiratory mask 1600 (e.g., for use with a CPAP or BiPAP system) according to one embodiment. The respiratory mask 1600 may include one or more antimicrobial medical device, for example a flexible face cushion or membrane, a frame, a housing, a forehead pad, an elbow, a cushion chip, and/or a swivel. In some examples, an antimicrobial flexible face cushion or membrane, and/or one or more rigid components (e.g., a frame, a housing, an elbow, a cushion chip, and/or a swivel) may be formed by injection molding, e.g., as discussed above.

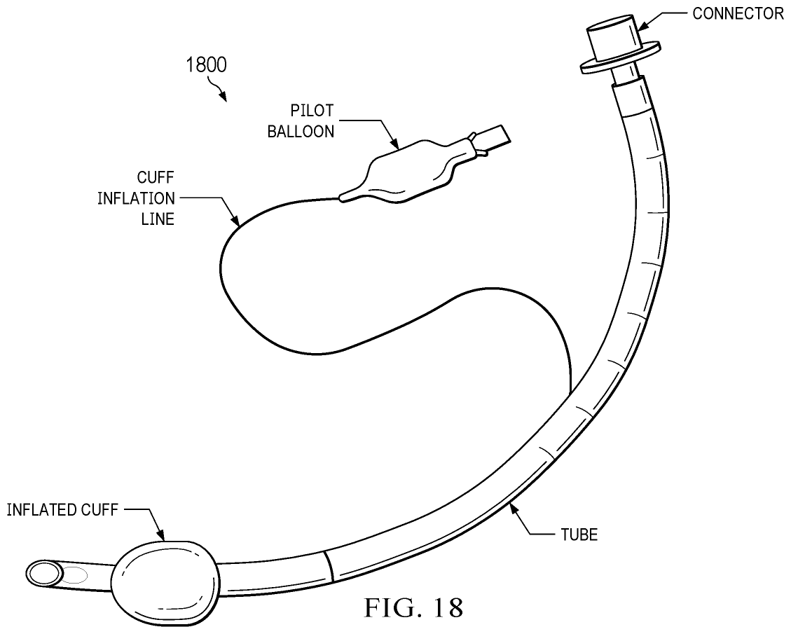

FIG. 17 shows an example nasal pillows respiratory interface 1700 (e.g., for use with a CPAP or BiPAP system) according to one embodiment. The nasal pillows respiratory interface 1700 may include one or more antimicrobial medical device, for example a tube assembly, a frame, nasal pillows, and/or a headgear or components of a headgear.

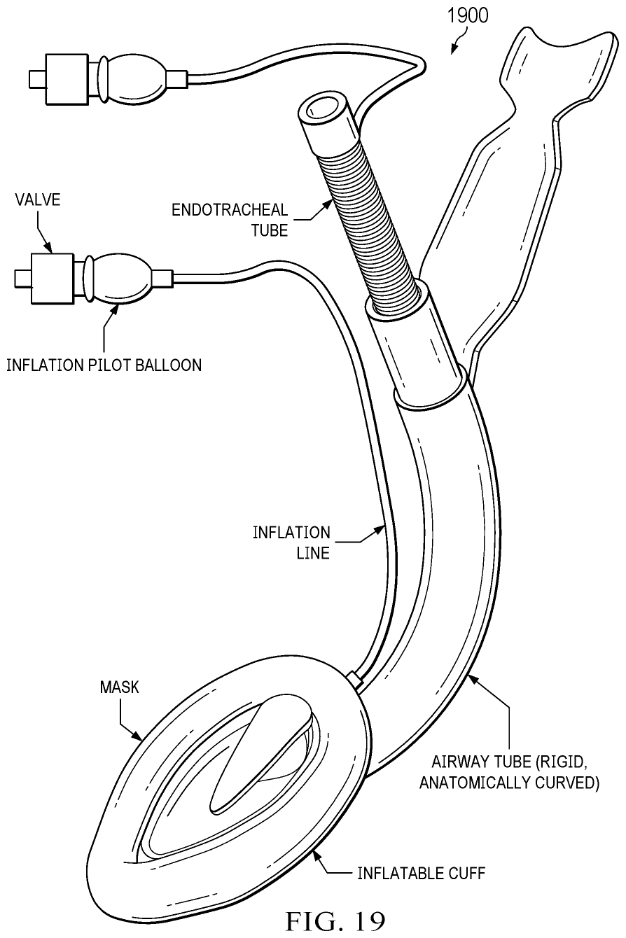

FIG. 18 shows an example endotracheal tube 1800 according to one embodiment. The endotracheal tube 1800 may include one or more antimicrobial medical device, for example nasal pillows, a frame, a tubing assembly, and/or headgear.

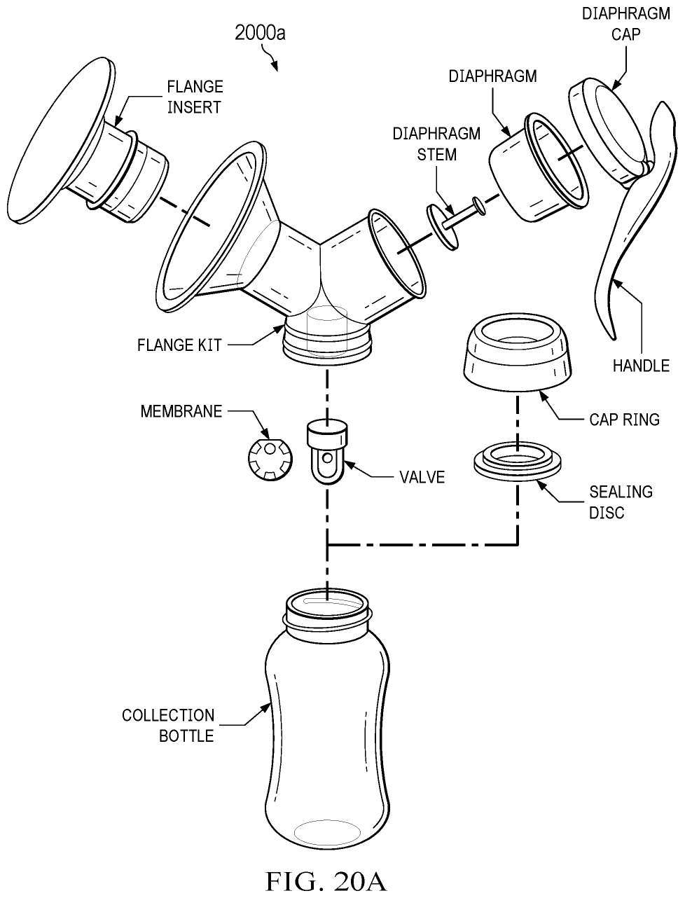

FIG. 19 shows an example laryngeal seal device 1900 according to one embodiment. The laryngeal seal device 1900 may include one or more antimicrobial medical device, for example an airway tube, a mask, an inflation line, an inflatable cuff, a pilot balloon, and/or a connector.

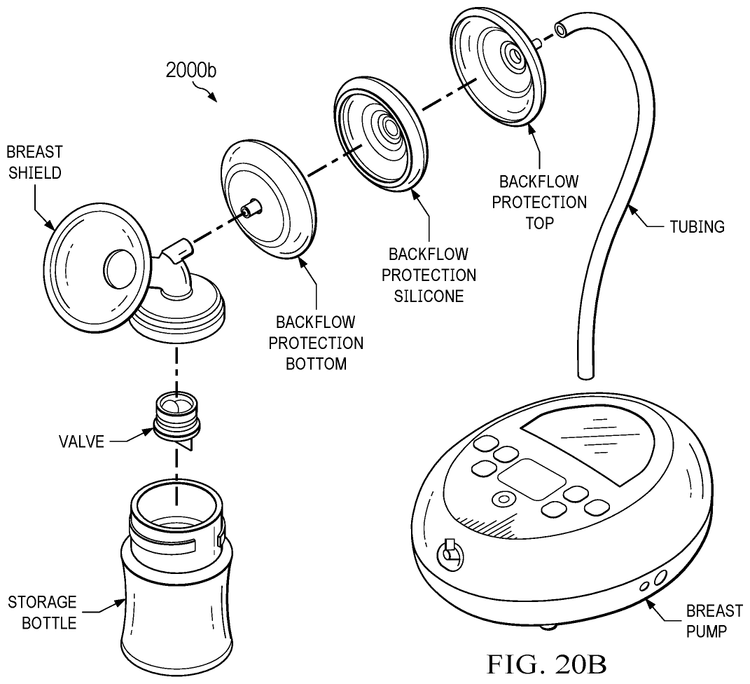

FIGS. 20A and 20B show example breast pump systems 2000a and 2000b according to example embodiments. Each breast pump system 2000a and 2000b may include one or more antimicrobial medical device, for example a collection bottle, a storage bottle, a sealing disc, a cap ring, a membrane, a gasket, a valve, a flange, a diaphragm, a diaphragm cap, a breast shield, a breast cushion, a backflow valve, and/or tubing.

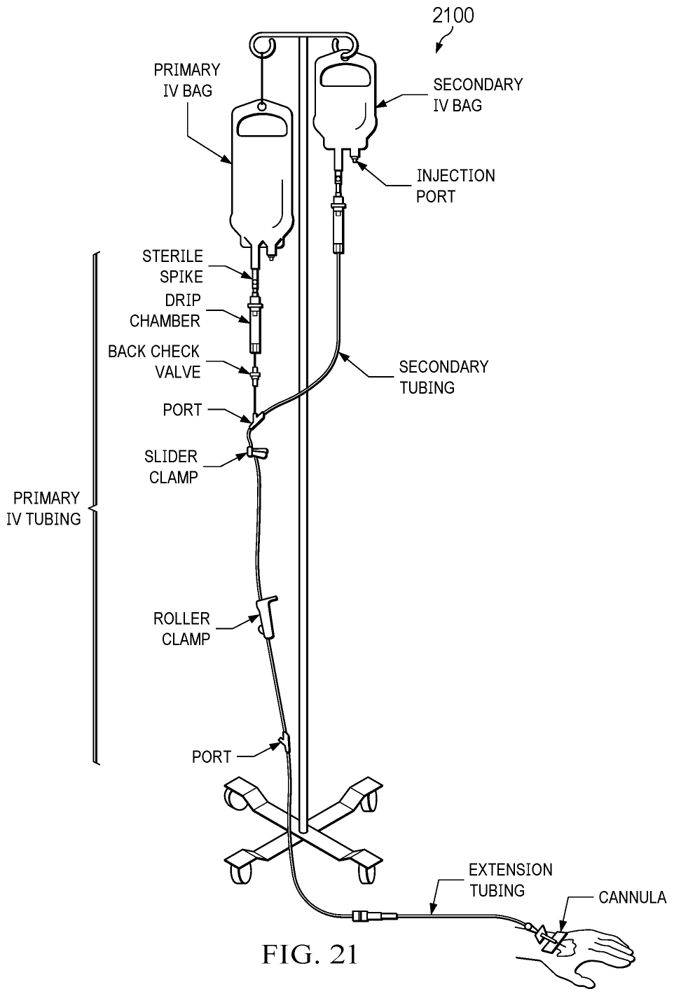

FIG. 21 shows an example intravenous (IV) therapy system 2100, according to one embodiment. The IV therapy system 2100 may include one or more antimicrobial medical device, for example IV bag(s), tubing, an injection port, clamps, a canula, port(s), and/or valve(s).

Although the disclosed embodiments are described in detail in the present disclosure, it should be understood that various changes, substitutions and alterations can be made to the embodiments without departing from their spirit and scope.

1. A method, comprising:
forming an antimicrobial blend including a quantity of an antimicrobial additive combined with a quantity of polymer; and
forming a medical device with the antimicrobial blend, wherein a surface of the medical device exhibits antimicrobial properties.

2. The method of claim 1, wherein:
the medical device comprises a conduit configured to carry a fluid; and
forming the medical device with the antimicrobial blend comprises using an extruder to extrude the conduit from the antimicrobial blend, wherein an interior surface of the extruded conduit exhibits antimicrobial properties.

3. The method of claim 1, wherein forming the medical device with the antimicrobial blend comprises using a molding system to perform a molding process to form the medical device from the antimicrobial blend.

4. The method of claim 1, wherein forming the medical device with the antimicrobial blend comprises using an additive manufacturing machine to print the medical device from the antimicrobial blend.

5. The method of claim 1, wherein the antimicrobial additive comprises at least one of (a) silver phosphate glass oxide or (b) copper oxide.

6. The method of claim 1, wherein the antimicrobial additive comprises silver, silver oxide, copper, or copper phosphate glass oxide.

7. The method of claim 1, wherein a percentage-by-weight of the antimicrobial additive relative to the polymer is in the range of 0.5% to 10%.

8. The method of claim 1, wherein a percentage-by-weight of the antimicrobial additive relative to the polymer is at least 2.5%.

9. The method of claim 1, wherein a percentage-by-weight of the antimicrobial additive relative to the polymer is in the range of 2.5% to 10%.

10. The method of claim 1, wherein a percentage-by-weight of the antimicrobial additive relative to the polymer is in the range of 3.5% to 5%.

11. The method of claim 1, wherein the polymer comprises a thermoplastic polymer.

12. The method of claim 1, wherein the polymer comprises polyethylene, polypropylene, polycarbonate, or silicone.

13. The method of claim 1, wherein:
the antimicrobial additive comprises silver phosphate glass oxide; and
the percentage-by-weight of the silver phosphate glass oxide relative to the polymer is in the range of 2.5% to 10%.

14. A medical device, comprising:
an antimicrobial structure comprising a mixture of:
a polymer; and
an antimicrobial additive comprising at least one of silver phosphate glass oxide or copper oxide, silver, silver oxide, copper, or copper phosphate glass oxide;
wherein a surface of the antimicrobial structure exhibits antimicrobial properties.

15. The medical device of claim 14, wherein a percentage-by-weight of the antimicrobial additive relative to the polymer is in the range of 0.5% to 10%.

16. The medical device of claim 14, wherein a percentage-by-weight of the antimicrobial additive relative to the polymer is in the range of 2.5% to 10%.

17. The medical device of claim 16, wherein the surface of the antimicrobial structure provides at least 99.85% reduction (log 2 efficacy) of E. coli bacteria and at least 99.99% reduction (log 4 efficacy) of S. aureus bacteria.

18. The medical device of claim 14, wherein a percentage-by-weight of the antimicrobial additive relative to the polymer is in the range of 3.5% to 10%.

19. The medical device of claim 18, wherein the surface of the antimicrobial structure provides at least 99.99% reduction (log 4 efficacy) of both E. coli bacteria and S. aureus bacteria.

20. The medical device of claim 14, wherein:
the antimicrobial additive comprises silver phosphate glass oxide; and
the percentage-by-weight of the silver phosphate glass oxide relative to the polymer is in the range of 3.5% to 10%.

21. The medical device of claim 14, wherein the antimicrobial structure comprises a conduit for carrying a fluid toward or away from a user.

22. The medical device of claim 21, wherein the conduit comprises a component of a CPAP system, BiPAP system, or ventilator system.

23. The medical device of claim 14, wherein the antimicrobial structure comprises a respiratory mask or a component of a respiratory mask.

24. A medical device, comprising:
an antimicrobial structure comprising a mixture of:
polyethylene, polypropylene, polycarbonate, or silicone; and
an antimicrobial additive comprising silver phosphate glass oxide or copper oxide;
wherein a percentage-by-weight of the silver phosphate glass oxide relative to the polymer is in the range of 2.5% to 10%; and
wherein a surface of the antimicrobial structure provides at least 99.85% reduction (log 2 efficacy) of E. coli bacteria and at least 99.99% reduction (log 4 efficacy) of S. aureus bacteria.
